(19) United States
(12) Patent Application Publication
(10) Pub. No.: US 2010/0280554 A1
(43) Pub. Date: Nov. 4, 2010

(54) Novel V construct and method of spinal stabilization after transforminal lumbar interbody fusion using the construct

(76) Inventor: Rahul Vaidya, Tecumseh (CA)

Correspondence Address: David W. Schumaker, 2983 Waterview Dr, Rochester Hills, MI, 48309, US

(21) Appl. No.: 12/387,243

(22) Filed: May 1, 2009

(51) Int. Cl. A61B 17/88; A61B 17/70; A61B 17/86

(52) U.S. Cl. 606/279; 606/246; 606/305

(57) ABSTRACT

A novel spinal stabilization instrumentation construct and method for use after transforaminal lumbar interbody fusion. The instrumentation construct consists of single pedicle screw attached to a translaminar screw with a rod (V construct). The V construct is superior to unilateral pedicle screws (UPS) construct in all movements except right lateral bending and left axial rotation and compares favorably with the unilateral pedicle screws with a translaminar screw. It provides bilateral fixation with minimal implant load and preserves the anatomical integrity of the superior facet joint. The construct offers significant advantages of reduction in surgical time, duration of hospitalization, cost of implants and a possibility of decreased incidence of adjacent segment disease.

### FIELD OF THE INVENTION

The present invention relates generally to transforaminal lumbar interbody fusion surgery and more specifically a novel V construct for stabilization of the spine after transforaminal lumbar interbody fusion surgery.

### BACKGROUND

Since the introduction of the transforaminal lumbar interbody fusion (TLIF) approach as an alternative to the posterior lumbar interbody fusion (PLIF) and anterior lumbar interbody fusion (ALIF) techniques in the early 1980s, the procedure has gained significant popularity. This development might be explained by several advantages attributed to the TLIF procedure. While accessing the disc transforaminally, there is no need to retract the thecal sac to either side to access the intervertebral space. This maneuver is responsible for the high rates of postoperative nerve root lesions and dural tears, which are reported in patients treated with PLIF. Furthermore, the bypassing of the dural sac in the TLIF technique allows instrumentation of levels above L-3, which are said to be dangerous to treat surgically with PLIF because the conus medullaris is at risk in these levels. Biomechanically, the preservation of the longitudinal ligaments is thought to preserve stability, to prevent implant dislocation, and to place compression on an adequately sized intervertebral implant. Additionally, this access avoids resection of the ligamentum flavum as well scarring of the spinal canal.

TLIF has become a well established method of surgical treatment for lumbar spinal disorders such as spondylolisthesis and discogenic back pain. The TLIF procedure allows a single exposure access for interbody fusion and posterior instrumentation. It preserves the anterior longitudinal ligament and a major portion of the posterior ligament complex with minimal compromise of spinal stability. However, following TLIF the spine needs to be instrumented since studies have shown that mechanically stable spines have a greater chance of fusion. Instrumentation enhances the stiffness of the fused segment and improves fusion rates lowering the chances of nonunion at the graft site. The stiffness of the spine increases 2.4 times following the use of instrumentation. In an attempt to increase the stability of the spine the volume of instrumentation has often been disproportionately increased. This may lead to injury to muscles, ligaments, and adjacent facet joints causing impaired fusion.

Bilateral transpedicular instrumentation using bilateral pedicle screws is routinely used in conjunction with an interbody cage to provide additional stability until the formation of a fusion mass. Their consistent use points towards their efficacy and reliability in outcomes. Fusion rates with bilateral pedicle screw instrumentation are reported to be in the range of 90 to 100%. However, pedicle screws have been associated with neural and vascular damage during placement along with extensive soft tissue injury and increased blood loss during the procedure. Further, extensive paraspinal muscle retraction is required for their insertion with consequent increased infection rates and muscle injury.

Unilateral pedicle screw reduces the extent of tissue injury and blood-loss. Lesser soft tissue dissection allows for early recovery and rehabilitation of the patient. It leads to less postoperative pain and reduced surgical time. However, it has been established that unilateral constructs may lead to increased rates of hardware failure. Though the degree of stability required for spinal fusion is unknown, unilateral pedicle screw constructs have been reported to result in inadequate spine stiffness with inferior results. Presence of coupled motions due to asymmetry and inability to provide enough rigidity are drawbacks of unilateral internal fixation. Further, considerable off-axis rotational motion was detected in the unilateral pedicle screw construct causing significant reduction of stiffness. Additionally, unilateral compression using the pedicle screws may cause undesirable scoliosis.

More recently, translaminar facet screws have also been utilized, mostly bilaterally, as a supplement to anterior lumbar interbody fusion. It has been reported that biomechanically the translaminar facet screw fixation may provide stability comparable to that of a pedicle screw system. During a TLIF procedure for discogenic back pain, contralateral facet and lamina are usually kept intact. Thus, a translaminar facet screw is inserted instead of a pedicle screw-rod construct on the side contralateral to TLIF. Unilateral pedicle screws with a translaminar screw are gaining acceptance. The unilateral pedicle screws and contralateral translaminar screw construct minimizes the biomechanical drawbacks of unilateral pedicle screws. The rate of neurological deficit and cerebrospinal fluid leakage during translaminar screw insertion is reported to be one half to one quarter that associated with pedicle screws. Wound infection rates, among cases of translaminar screws are also described as being one tenth that of pedicle screws. The use of unilateral pedicle screws and a translaminar screw allows fusion to be done with little muscle stripping often associated with posterolateral fusion. Yet, it accomplishes a 270 degree fusion with interbody and posterior fusion of the contralateral posterior spinal elements.

However, placement of the superior pedicle screw (of the unilateral pedicle screw and rod construct) can damage the inferior facet of the adjacent segment altering the facet load bearing capability and has been implicated as a risk factor in the development of adjacent level disease (ASD). Further, the capsular integrity of the superior facet joint is also at risk during placement. A study of literature on ASD reported that addition of instrumentation to fusion appears to be a risk for early development of ASD. Further, the interval of occurrence of ASD appeared to be shorter with instrumented fusions.

Thus, minimally invasive techniques and instrumentation constructs that reduce implant load are needed in the art to minimize soft tissue injury and retain the facetal anatomy. Significant reductions of operative time, duration of hospitalization and costs will be the benefits of these techniques.

### SUMMARY OF THE INVENTION

The present invention relates to an instrumentation construct for stabilizing the superior and inferior vertebrae in a spine after transforaminal lumbar interbody fusion surgery. The instrumentation construct consists of a single pedicle screw, a single rod, and a single translaminar screw. The single pedicle screw and the single translaminar screw are interconnected with the single rod. The single translaminar screw may include a tulip head connector. The single translaminar screw may be a polyaxial pedicle screw. The single pedicle screw, single rod and single translaminar screw may be composed of titanium.

The instant invention also includes an improvement in the transforaminal lumbar interbody fusion surgical method, in that the improvement is comprised in stabilization of the superior and inferior vertebrae using the inventive instrumentation construct of the present invention. The single pedicle screw is attached into the pedicle of the inferior vertebra on the ipsilateral side of the spine. The single translaminar screw is inserted into the contralateral translaminar position of the spine. The single translaminar screw is introduced into the junction of the spinous process and lamina on the ipsilateral side of the superior vertebra, passes through the contralateral facet joint, and terminates in the base of the contralateral pedicle of the inferior vertebra.

### BRIEF DESCRIPTION OF THE DRAWINGS

FIG. 1 is a schematic depiction of a typical prior art BPS fixation system in place in a spine;

FIG. 2 is a schematic depiction of a typical prior art UPS fixation system in place in a spine;

FIG. 3 is a schematic depiction of a typical prior art UPS+TS fixation system in place in a spine;

FIG. 4 is a schematic depiction of the inventive V construct of the instant invention;

FIGS. 5, 6, 7a and 7b show exploded views of three different types of pedicle screws which may be useful in the method of the present invention;

FIG. 8 is a graphical representation of the results of flexion/extension testing for, an intact spine, the prior art constructs and the V construct of the present invention;

FIG. 9 is a graphical representation of the results of left/right lateral bending testing for, an intact spine, the prior art constructs and the V construct of the present invention; and

FIG. 10 is a graphical representation of the results of left/right axial rotation testing for, an intact spine, the prior art constructs and the V construct of the present invention.

### DETAILED DESCRIPTION OF THE INVENTION

The inventor has designed a novel V construct that reduces implant load in lumbar interbody fusion to minimize soft tissue injury and retain the facetal anatomy. The V construct eliminates the placement of the superior pedicle screw in single level lumbar fusion. The V construct is most similar to the unilateral pedicle screws with a translaminar screw construct of the prior art. The V construct eliminates the superior pedicle screw. The support rod connects the inferior pedicle screw and the translaminar screw. The device provides bilateral fixation and does not put at risk the integrity of the superior facet joint.

The inventive V construct and the associated surgical spinal stabilization method using the V construct offer several advantages over the constructs of the prior art first, the cost of a traslaminar facet screw is substantially lower than a pedicle screw-rod construct. A facet screw typically costs about $200 versus pedicle screws and rods, which cost around $1,000 apiece. A second advantage of facet screw insertion is it does not put the integrity of the superior facet joint at risk. Thus, the inventive V construct offers significant advantages of reduction in surgical time, cost of implants and a possibility of decreased incidence of adjacent segment disease.

The inventive V construct was compared to prior art fixation constructs for single level posterior lumbar fusions. The prior art constructs compared with the instant invention were: 1) bilateral pedicle screws (BPS); 2) unilateral pedicle screws (UPS); and 3) unilateral pedicle screws combined with a contralateral translaminar screw (UPS+TS).

As used herein, the terms ipsilateral and contralateral mean the side of the spine on which the TLIF surgery has been performed and the side of the spine opposite the TLIF surgery, respectively. The terms “superior vertebra” and “inferior vertebra” mean the vertebra above and below the site TLIF disk removal, respectively. The terms “translaminar screw” and “translaminar facet screw” are used interchangeable and have the same meaning.

### Bilateral Pedicle Screw (BPS) Fixation

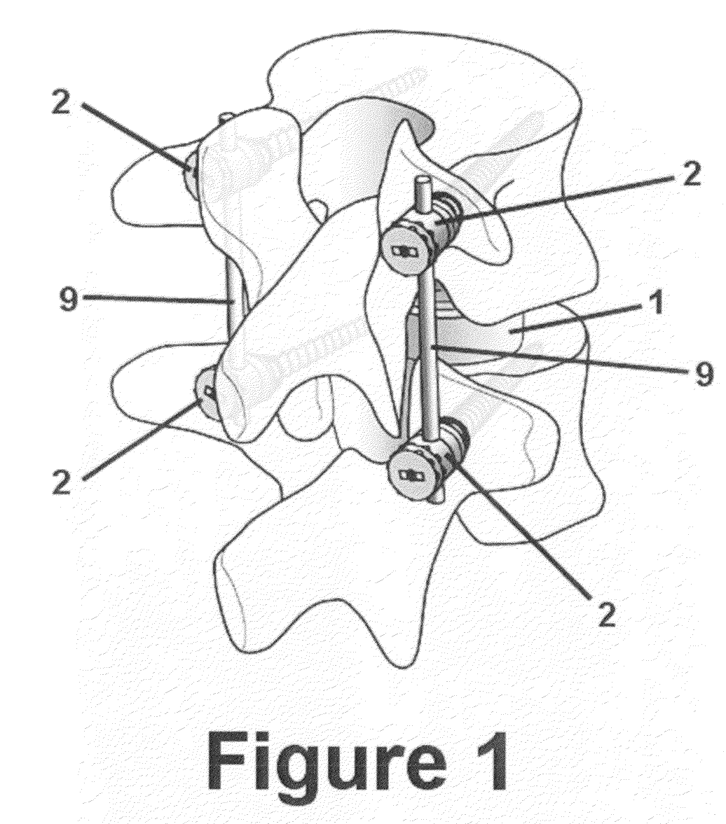

FIG. 1 is a schematic depiction of a typical prior art BPS fixation system in place in a spine. Two sets of pedicle screws 2 connect two longitudinally oriented rods 9. The two sets of two pedicle screws 2 and rod 9 are attached to the pedicles of the superior and inferior vertebra on the both the ipsilateral and contralateral sides of the spine. Also visible is the intervertebral spacer 1.

### Unilateral Pedicle Screw (UPS) Fixation

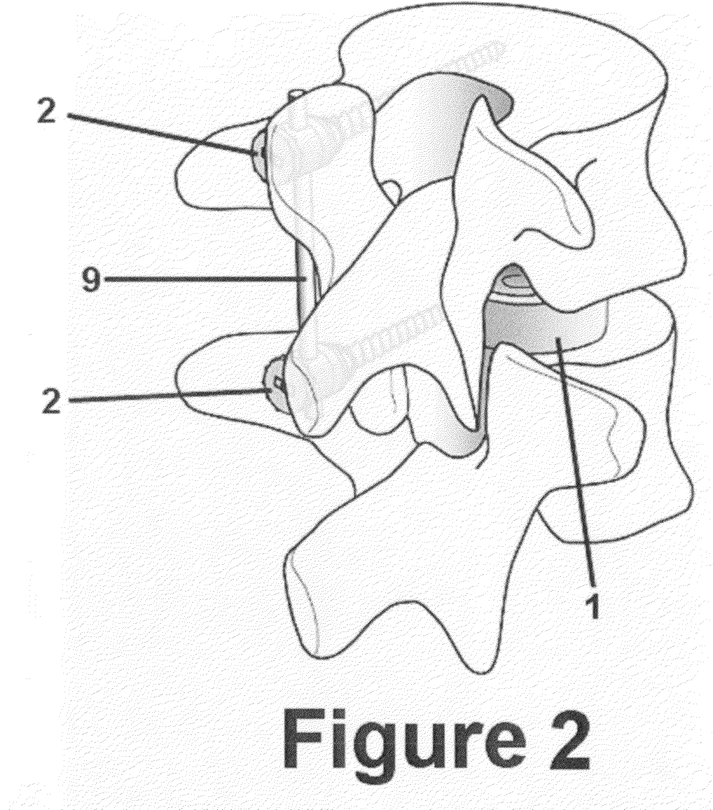

FIG. 2 is a schematic depiction of a typical prior art UPS fixation system in place in a spine. A single set of pedicle screws 2 connect a single longitudinally oriented rod 9. The set of two pedicle screws 2 and rod 9 are attached to the pedicles of the superior and inferior vertebra on the ipsilateral side of the spine only. Also visible is the intervertebral spacer 1.

### Unilateral Pedicle Screw Plus Translaminar Screw (UPS+TS) Fixation

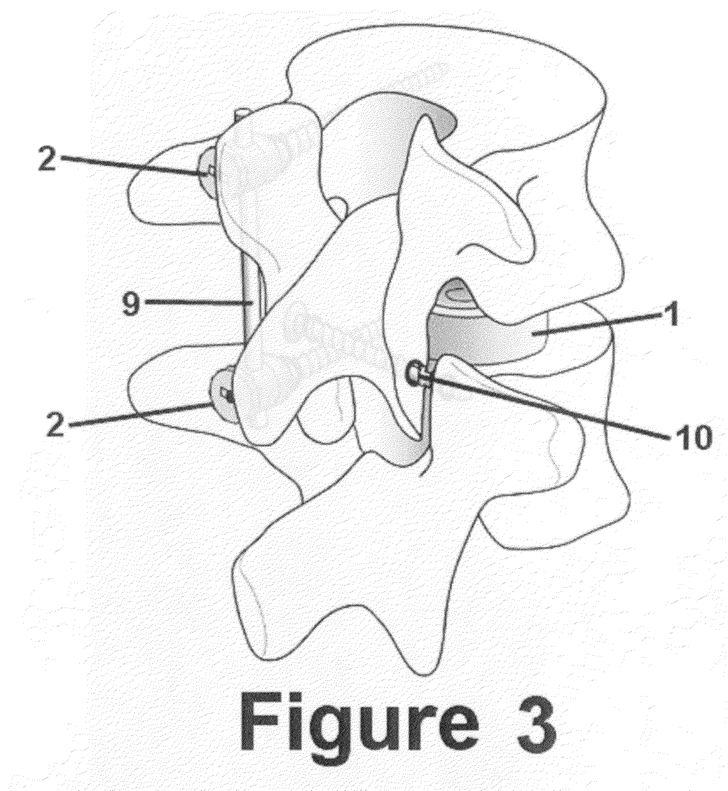

FIG. 3 is a schematic depiction of a typical prior art UPS+TS fixation system in place in a spine. A single set of pedicle screws 2 connect a single longitudinally oriented rod 9. The set of two pedicle screws 2 and rod 9 are attached to the pedicles of the superior and inferior vertebra on the ipsilateral side of the spine. In addition to the two pedicle screws/rod fixation, the construct includes a contralateral translaminar facet screw 10. Also visible is the intervertebral spacer 1.

Single Pedicle Screw/Rod/Translaminar Screw (V construct) Fixation

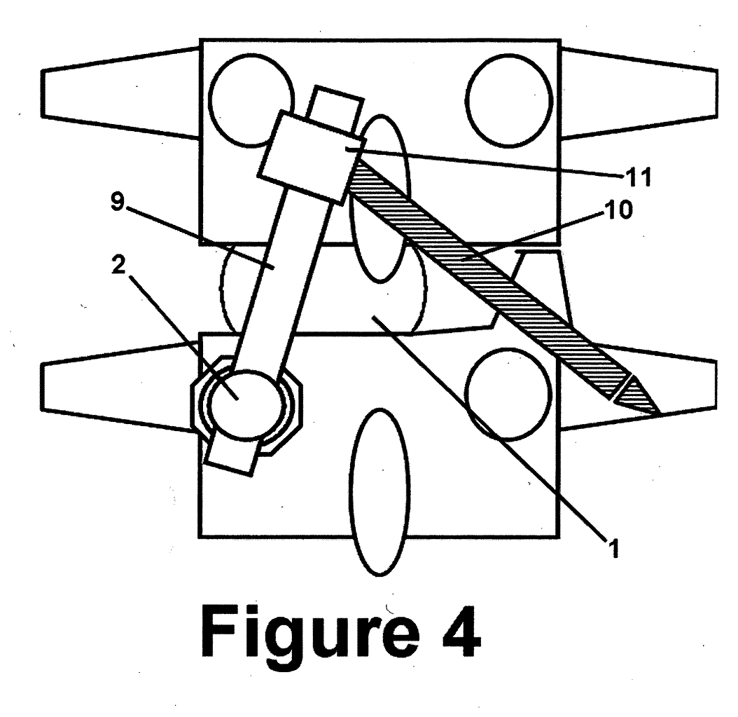

FIG. 4 is a schematic depiction of the inventive V construct of the instant invention. A single pedicle screw 2 and a single translaminar facet screw 10 connect to a single rod 9. The single pedicle screw 2 is attached to the pedicle of the inferior vertebra on the ipsilateral side of the spine. The translaminar screw 10 is in a contralateral translaminar position. The entry point of the screw is at the junction of the spinous process and lamina of the ipsilateral side of the superior vertebra. The translaminar screw passes through the contralateral facet joint. The exit point is the base of the contralateral pedicle of the inferior vertebra. This translaminar technique maximizes the strength and length of the screw both proximal and distal to the facet joint. The rod 9 is attached to the pedicle screw 2 on one end and is attached to the translaminar screw 10 on the other end. The translaminar screw is preferably designed as a polyaxial pedicle screw (4.0-5.0 mm preferred) with a tulip head connector 11 allowing fixation of the inferior pedicle screw 2 to the translaminar screw 10 with a rod 9. Also visible is the intervertebral spacer 1.

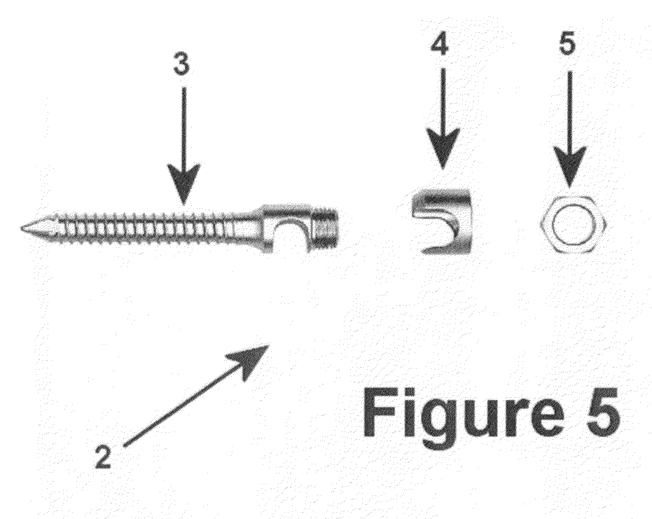

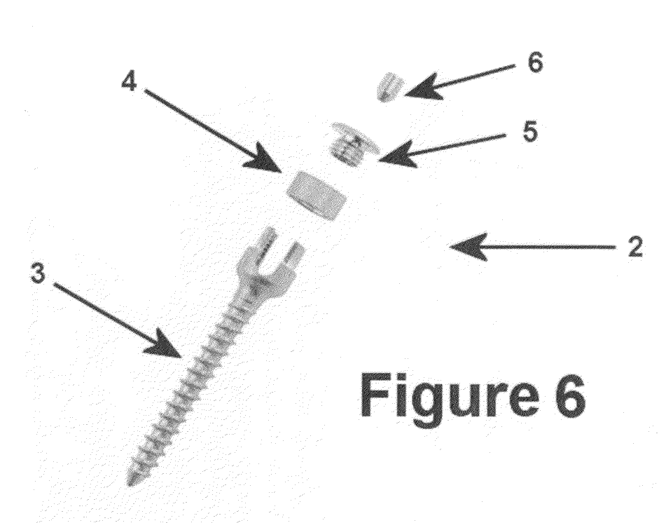

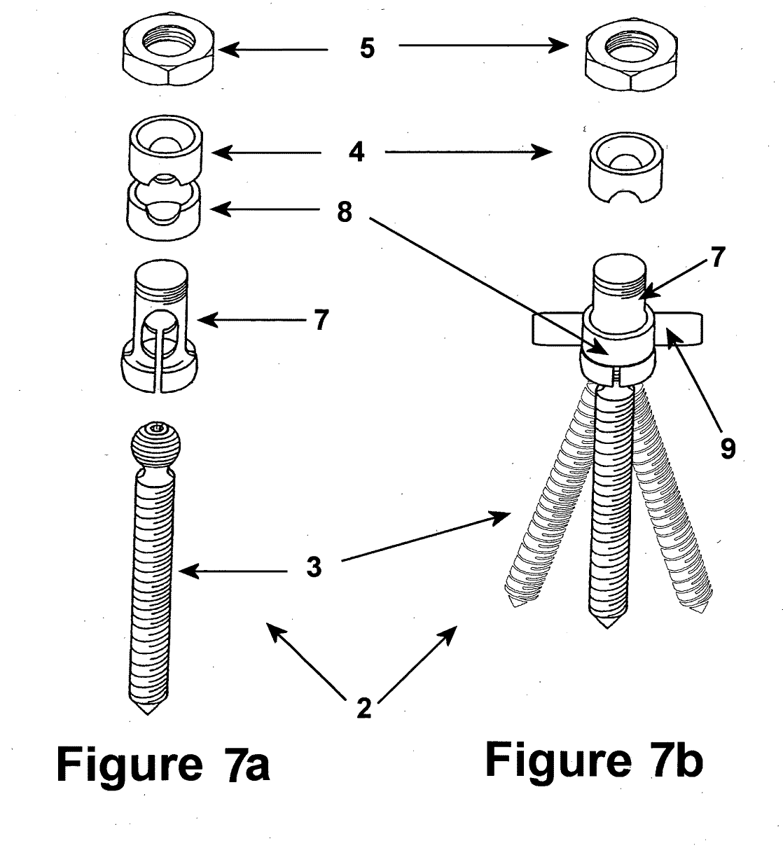

FIGS. 5, 6, 7a and 7b show exploded views of three different types of pedicle screw 2 which may be useful in the method of the present invention. Referring to FIG. 5, the separate parts of a side-opening pedicle screw can be seen. Specifically shown are the screw 3, the sleeve 4 and the nut 5. In use, the sleeve 4 and nut 5 are placed over the screw 3 and hold a rod in the cylindrical opening formed by the mating of the screw 3 and the sleeve 4. Turning to FIG. 6, a tulip head type of pedicle screw 2 is seen. In this variety, there is still a screw 3, a sleeve 4 and a nut 5, but there is also a set screw 6 which helps to hold a rod in the opening between the screw 3 and the sleeve 4. Finally, FIGS. 7a and 7b show a polyaxial pedicle screw having a swivel joint. Once again this variety of pedicle screw has a screw 3, a sleeve 4 and a nut 5, but this type also has a mechanism consisting of a swivel clamp 7 and a swivel clamp collar 8. This added hardware allows the head of the pedicle screw to swivel somewhat independently from the screw 3. Thus this swivel head allows for ease of fit to a rod 9 without the requirement for excessive rod contouring. Preferably the pedicle screw, translaminar facet screw and the fixation rod are formed of titanium.

### Example/Testing

Seven fresh ligamentous human lumbar spine specimens (L2-L5) were tested on a spine motion simulator. A pure moment load control protocol was used for testing for flexion-extension, lateral bending and axial rotation. After intact testing the specimens underwent a standard transforaminal interbody fusion at L3-L4 using a polyetheretherketone (PEEK) cage. Each specimen was then tested with the above prior art fixation constructs/systems and compared with the inventive V construct/system.

### Specimen Preparation

To compare the prior art constructs/fixation systems and the present V construct, seven fresh ligamentous human lumbar spine specimens (L2-L5) were stored in double plastic bags at −200 C. The spines were radiographed in both the anteroposterior and lateral planes to ensure the absence of fractures, deformities and any metastatic disease. Before testing, the specimens were thawed at room temperature for 10 hours and then cleaned to remove any extraneous fat, muscle, and tissue. The L2 and L5 vertebrae of each specimen were then potted firmly in a mixture of BONDO® brand auto body filler (3M BONDO Corporation) and Bondo HOME SOLUTIONS® brand all purpose fiberglass resin (3M BONDO Corporation). Three markers were secured rigidly to each vertebral body to track its motion with the Optotrak Certus® motion capture system (Northern Digital, Inc).

### Test Protocol

The prepared specimens were mounted on a custom built 6 degree of freedom spine motion simulator designed to apply bending moments in the physiologic planes of the spine. A pure moment load control protocol was used for testing with moments of ±8 Nm used for flexion-extension, lateral bending and axial rotation. During testing the specimens were kept moist with 0.9% saline solution. After intact testing the specimens underwent a standard TLIF at L3-L4. A polyetheretherketone (PEEK) cage was used as an interbody spacer (Globus Medical, Inc., Audubon Pa.). Range of Motion (ROM) data was acquired for each construct under the same loading conditions as with the intact specimen.

The average ROM for each construct was compared with the intact spine ROM as control (100%). One Way Single Factor ANOVA was used for analysis, comparing the surgical constructs to intact with significance at p≦0.05.

### Results:

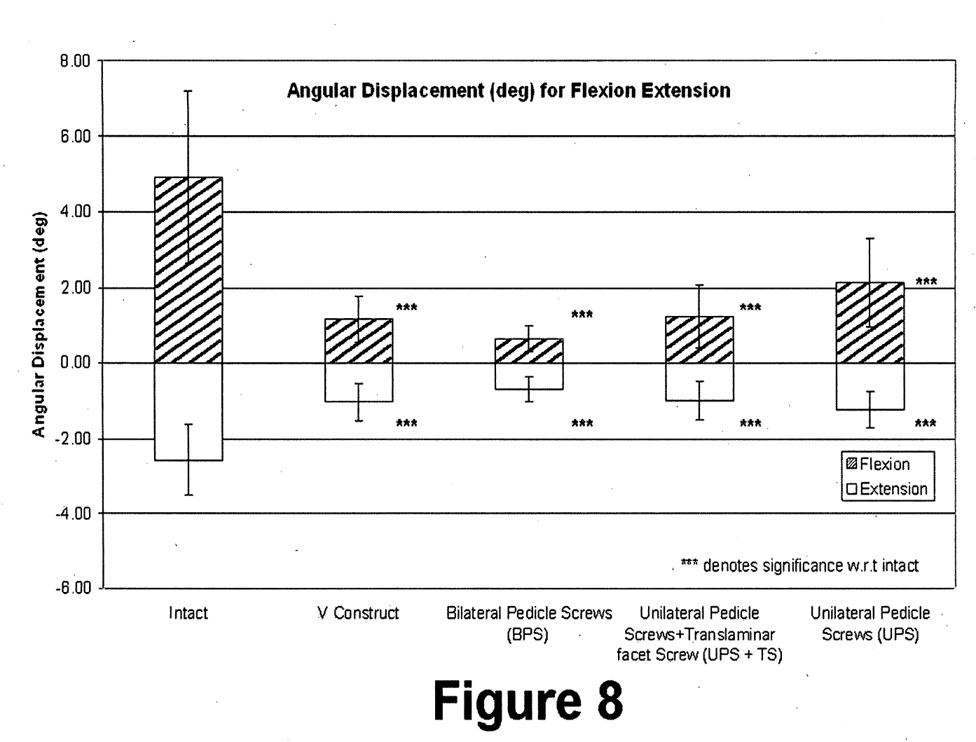

FIG. 8 is a graphical representation of the results of flexion/extension testing for, an intact spine, the prior art constructs and the V construct of the present invention. In flexion, the V construct exhibited a significant decrease in motion, 76% (p=0.001) compared to the intact spine. The BPS construct reduced flexion by 87% (0.00034) and the UPS by 57% (p=0.013). The UPS+TS construct caused a 75% (p=0.0056) motion reduction. In extension the reduction values were: V construct 60% (p=0.0026), BPS 73% (p=0.00034), UPS 52% (p=0.0062), UPS+TS 62% (p=0.0081).

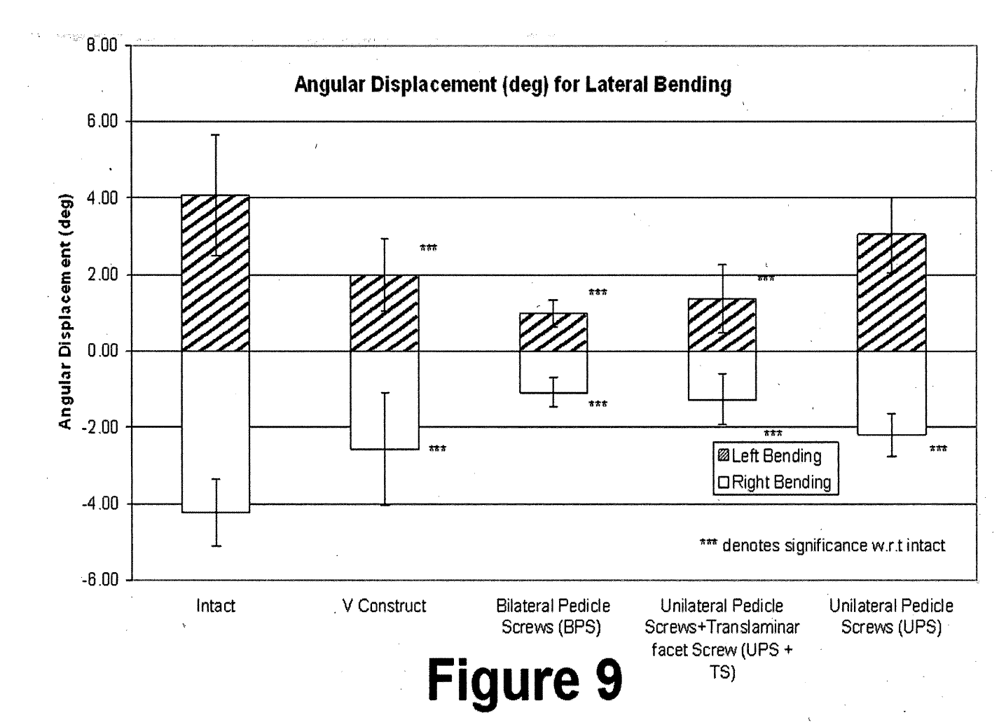

FIG. 9 is a graphical representation of the results of left/right lateral bending testing for, an intact spine, the prior art constructs and the V construct of the present invention. In left lateral bending, there was a decrease in motion of 51% (p=0.011) with the V construct compared with the intact spine. The BPS, UPS and the UPS+TS constructs reduced motion by 75% (p=0.00032), 40% (p=0.117) and 66% (p=0.008) respectively. There was a decrease in motion in right lateral bending for all the surgical constructs with values of 39% (p=0.047) for the V construct, 74% (p=1.41 E−06) for BPS, 70% (p=0.00028) for UPS+TS and 48% (p=0.036) for UPS constructs.

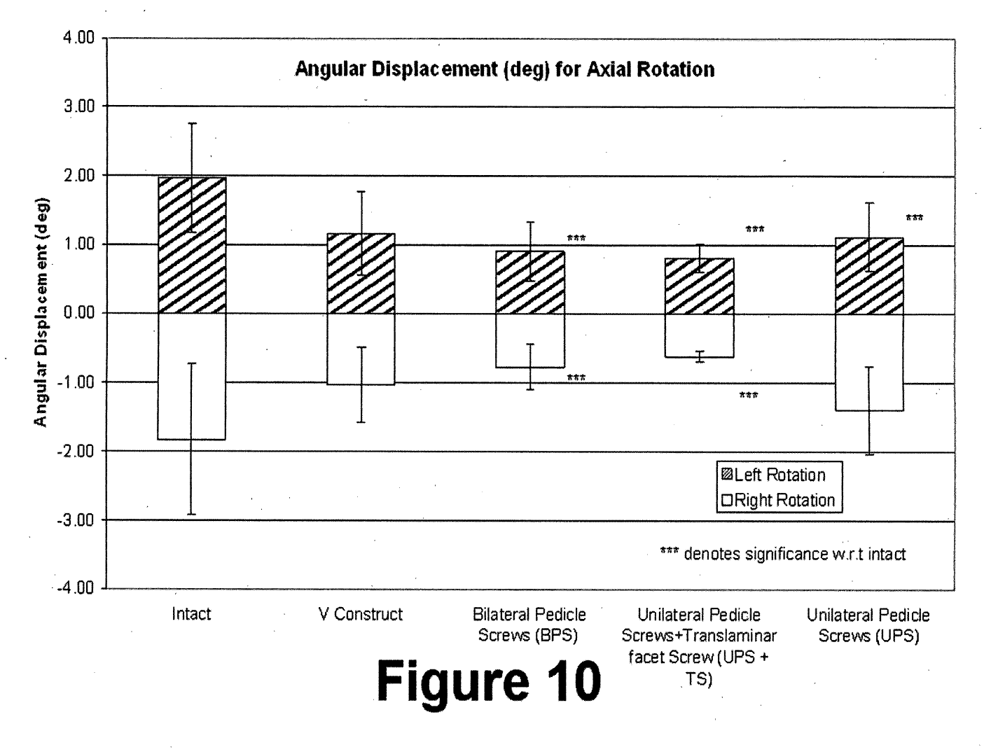

FIG. 10 is a graphical representation of the results of left/right axial rotation testing for, an intact spine, the prior art constructs and the V construct of the present invention. In left axial rotation the range of motion in the V construct and UPS construct decreased by 41% (p=0.07) and 44% (p=0.01) respectively. Range of motion in BPS and UPS+TS constructs decreased compared to the intact and stood at 53% (p=0.0088) and 58% (p=0.05) respectively. In right axial rotation, the trend was similar with the V construct showing a decrease in range of motion of 43% (p=0.171) and UPS construct showing a decrease of 26% (p=0.365). These values for BPS and UPS+TS constructs were 57% (p=0.03) and 66% (p=0.01) respectively.

In both flexion and extension our construct compared favorably with the UPS+TS and was superior to UPS alone. Increased ROM with the V construct in right lateral bending may be attributed to the left sided TLIF that was performed in all specimens. Bone density may affect the stability of the implant. A limitation of the test results was the lack of bone density measurements while testing. However, plain radiographs of cadaveric specimens were evaluated for the presence of fractures, deformity and metastatic disease. The construct is indicated for single level fusion and has limited application due to the requirement of an intact lamina and facet on the side of the translaminar screw. The ideal indication would be a patient with unilateral radicular complaints requiring a single level spinal fusion. Patients with bilateral decompression may make it difficult to pass the translaminar screw.

In conclusion, the V construct is superior to unilateral pedicle screws (UPS) construct in all movements except right lateral bending and left axial rotation. It provides bilateral fixation with minimal implant load and preserves the anatomical integrity of the superior facet joint which may possibly reduce the incidence of adjacent segment disease. Significant reduction in operation time, duration of hospitalization and costs of implants are the other advantages which this novel construct offers.

1. In a transforaminal lumbar interbody fusion surgical method, the improvement comprising the step of:
stabilizing the superior and inferior vertebrae using an instrumentation construct consisting of a single pedicle screw, a single rod and a single translaminar screw.

2. The method of claim 1, wherein said step of stabilizing the superior and inferior vertebrae includes the step of attaching said single pedicle screw into the pedicle of the inferior vertebra on the ipsilateral side of the spine.

3. The method of claim 1, wherein said step of stabilizing the superior and inferior vertebrae includes the step of inserting said single translaminar screw into the contralateral translaminar position of the spine.

4. The method of claim 3, wherein said step of inserting said single translaminar screw comprises:
introducing the translaminar screw at the junction of the spinous process and lamina on the ipsilateral side of the superior vertebra;
passing the translaminar screw through the contralateral facet joint; and
terminating the translaminar screw in the base of the contralateral pedicle of the inferior vertebra.

5. The method of claim 1, wherein said step of stabilizing the superior and inferior vertebrae includes the step of interconnecting said single pedicle screw and said single translaminar screw using said single rod.

6. The method of claim 5, wherein said step of interconnecting said single pedicle screw and said single translaminar screw using said single rod includes the step of connecting said translaminar screw to said rod using a tulip head connector.

7. The method of claim 1, wherein said single translaminar screw is a polyaxial pedicle screw.

8. The method of claim 1, wherein said single translaminar screw is a 4.0-5.0 mm pedicle screw.

9. The method of claim 1, wherein said single translaminar screw includes a tulip head connector.

10. The method of claim 1, wherein said single translaminar screw is a polyaxial pedicle screw having a tulip head connector.

11. The method of claim 10, wherein said polyaxial pedicle screw and said tulip head connector are composed of titanium.

12. The method of claim 1, wherein said single pedicle screw, single rod and single translaminar screw are composed of titanium.

13. An instrumentation construct consisting of:
a single pedicle screw;
a single rod; and
a single translaminar screw;
said instrumentation construct designed for stabilizing the superior and inferior vertebrae in a spine after transforaminal lumbar interbody fusion surgery.

14. The instrumentation construct of claim 13, wherein said single pedicle screw and said single translaminar screw are interconnected with said single rod.

15. The instrumentation construct of claim 14, wherein said single translaminar screw includes a tulip head connector.

16. The instrumentation construct of claim 15, wherein said single translaminar screw is a polyaxial pedicle screw.

17. The instrumentation construct of claim 16, wherein said single transiaminar screw is a 4.0-5.0 mm pedicle screw.

18. The instrumentation construct of claim 13, wherein said single translaminar screw includes a tulip head connector.

19. The instrumentation construct of claim 13, wherein said single translaminar screw is a polyaxial pedicle screw having a tulip head connector.

20. The instrumentation construct of claim 13, wherein said single pedicle screw, single rod and single translaminar screw are composed of titanium.
